(19) United States
(12) Patent Application Publication
(10) Pub. No.: US 2025/0387609 A1
(43) Pub. Date: Dec. 25, 2025

(54) MEDICAL VALVE WITH REDUCED FRICTION

(71) Applicant: CAREFUSION 303, INC., San Diego, CA (US)

(72) Inventor: Raymond P. FEITH, Chino Hills, CA (US)

(21) Appl. No.: 19/249,917

(22) Filed: Jun. 25, 2025

Related U.S. Application Data
(60) Provisional application No. 63/664,093, filed on Jun. 25, 2024.

(51) Int. Cl. A61M 39/26; A61M 5/14; A61M 39/00; A61M 39/10

(52) U.S. Cl. CPC A61M 39/26; A61M 5/1413; A61M 2039/0081; A61M 2039/1038; A61M 2039/267

(57) ABSTRACT

Needle-free fluid connectors with one or more grooves on a center post to limit friction are disclosed. A fluid connector may include a housing with an inner chamber having an opening. A post is located inside the housing and includes an aperture and a lumen. The aperture is fluidly connected to the lumen. A deformable sleeve is located on the post. The post includes grooves that limit the surface contact between the post and the sleeve to restrict the friction between the post and the sleeve. The sleeve can be displaced to expose the aperture, which fluidly connects the opening and the lumen via the aperture.

### RELATED APPLICATION DATA

This application claims priority to U.S. Provisional Application No. 63/664,093, filed Jun. 25, 2024, the entire content of which is incorporated herein by reference.

### TECHNICAL FIELD

The present disclosure relates generally to medical fluid connectors and, more particularly, to neutral displacement needle-free connectors.

### BACKGROUND

Medical treatments often include the infusion of a medical fluid (e.g., a saline solution or a liquid medication) to patients using an intravenous (IV) catheter that is connected though an arrangement of flexible tubing and fittings, commonly referred to as an “IV set,” to a source of fluid, for example, an IV bag. Often, tubing or catheters are coupled or secured to each other to allow fluid communication between various portions of tubing or catheters.

Needle-free connectors, including neutral displacement needle-free connectors, offer a solution for providing medical fluid to patients. A needle-free connector assembly promotes fluid transmission between a medical fluid supply and a catheter line. The medical fluid supply and the catheter line are secured to respective luers. To transmit fluid, the luer connected to the medical supply overlaps a central post of the luer connected to the catheter line.

### SUMMARY

In accordance with at least some embodiments disclosed herein is the realization that an elastomeric valve in a needle-free connector, which must open when a luer or post is inserted and close when the luer or post is removed, may become stuck in the open position or be slow to return to the closed position. This scenario can result in medical fluid leakage and reduced medical delivery to a patient.

Aspects of the present disclosure provide a needle-free fluid connector that minimizes the risk of the elastomeric valve in a needle-free connector becoming stuck in the open position or being slow to return to the closed position. The needle-free fluid connector reduces the friction between the elastomeric valve and the luer or post to help the elastomeric valve return to its original state—i.e., the closed position.

Accordingly, aspects of the present disclosure provide a fluid connector comprising: a housing comprising a proximal end and a distal end, the housing comprising an inner chamber having an opening at the proximal end; a post extending from a distal portion of the inner chamber toward the proximal end and comprising an aperture, at a proximal portion of the post, and a lumen fluidly connected to the aperture; and a resilient sleeve extending around the post from the distal portion toward the proximal end such that the sleeve seals the opening and the aperture in a covered configuration and is compressed distally to unseal the opening and the aperture in an uncovered configuration, wherein the post comprises one or more grooves along at least a part of an exterior surface of the post that limits contact between the post and the sleeve.

In some embodiments, the post is a generally conical shape. In some embodiments, the post is a cannula.

In some embodiments, the grooves contain a lubricant that restricts friction between the post and the sleeve. In some embodiments, the grooves comprise undulating ridges on an exterior of the post that restrict friction between the post and the sleeve by limiting surface contact between the post and the sleeve. In some embodiments, the grooves extend longitudinally from the distal portion of the inner chamber toward the proximal portion of the post.

In some embodiments, the sleeve extends circumferentially around the post.

In some embodiments, extend circumferentially around the entire post. In some embodiments, the grooves extend about half an entire length of the post.

In some embodiments, in the uncovered configuration, a proximal end of the sleeve is displaced toward a distal end of the sleeve and the fluid connector is configured to permit fluid to be conducted through the aperture and the lumen between the opening at the proximal end and a distal opening at the distal end.

Some instances of the present disclosure provide a method for restricting friction in a fluid connector during delivery of a medical fluid, the method comprising: providing a housing, the housing comprising a proximal end, a distal end, and an inner chamber having an opening at the proximal end; providing a post extending from a distal portion of the inner chamber toward the proximal end and comprising an aperture, at a proximal portion of the post, and a lumen fluidly connected to the aperture; providing a resilient sleeve extending around the post from the distal portion toward the proximal end; and providing one or more grooves, along at least a part of an exterior surface of the post, to limit surface contact between the post and the sleeve; wherein the fluid connector is configured to form, by compressing the sleeve distally to unseal the opening and the aperture, a fluid pathway between the aperture and the lumen.

In some embodiments, providing one or more grooves comprises applying a lubricant to the grooves to restrict friction between the post and the sleeve.

In some embodiments, forming a fluid pathway comprises displacing a proximal end of the sleeve toward a distal end of the sleeve such that fluid may be conducted, through the aperture and the lumen, between the opening at the proximal end and a distal opening at the distal end.

Accordingly, the present application addresses several operational challenges encountered in prior needle-free connector assemblies that are susceptible to leaking.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates an IV set coupled to a patient, in accordance with aspects of the present disclosure.

FIGS. 2A and 2B illustrate perspective views of the fluid connector assembly in the uncoupled position and the coupled position, respectively, in accordance with aspects of the present disclosure.

FIG. 3 illustrates a cross-sectional view of the fluid connector assembly in the coupled position, in accordance with aspects of the present disclosure.

FIG. 4 illustrates a cross-sectional view of the fluid connector, in accordance with aspects of the present disclosure.

FIG. 5 illustrates a cross-sectional view of the post of the fluid connector, in accordance with aspects of the present disclosure.

FIG. 6 illustrates a flowchart showing a method for reducing friction in a fluid connector during delivery of a medical fluid, in accordance with aspects of the present disclosure.

### DETAILED DESCRIPTION

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

In accordance with some embodiments, the present disclosure includes various features and advantages of maintaining separation between a post and luer, thus minimizing the likelihood of damaging the post and/or the luer.

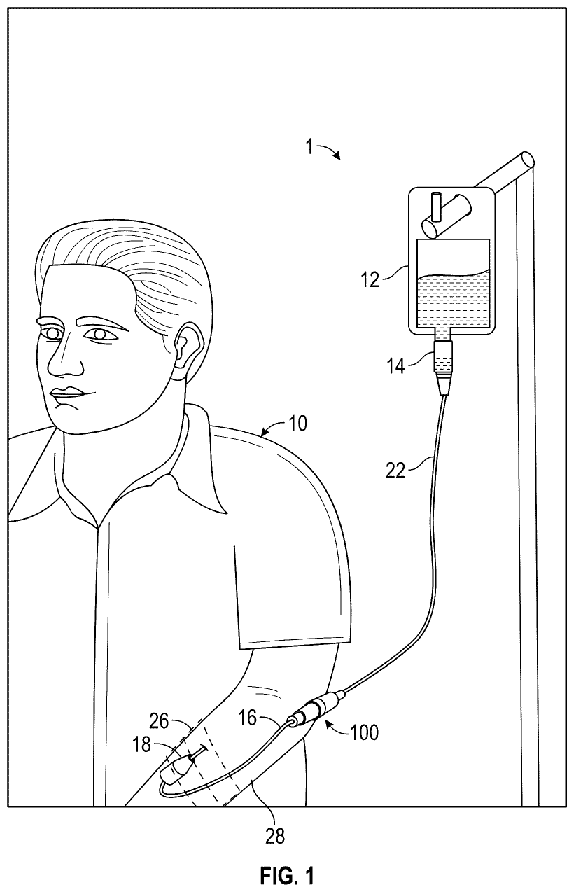

Referring now to the figures, FIG. 1 illustrates an IV set 1 coupled to a patient 10, in accordance with aspects of the present disclosure. The IV set 1 includes a medical fluid bag 12, a drip chamber 14, and tubing 22. The tubing 22 extends between the drip chamber 14 and a fluid connector assembly 100 of the IV set 1. The fluid connector assembly 100 is designed for use in medical applications, such as the IV set 1 (shown in FIG. 1) as well as other IV medical fluid delivery applications using catheters, including peripheral intravenous catheters (PIVC), as non-limiting examples.

To resist unintended dislodgement or disconnection of the tubing 16 or the catheter 18 from the patient, tape 26 is placed over the tubing 16 and the catheter 18, so that the tape 26 engages the tubing 16, the catheter 18, and the patient 10.

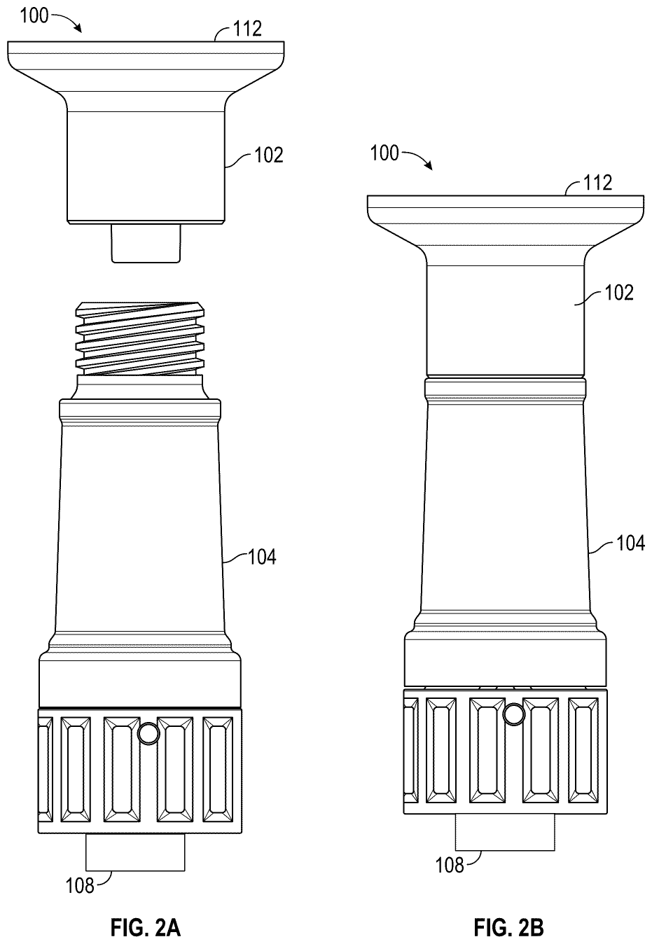

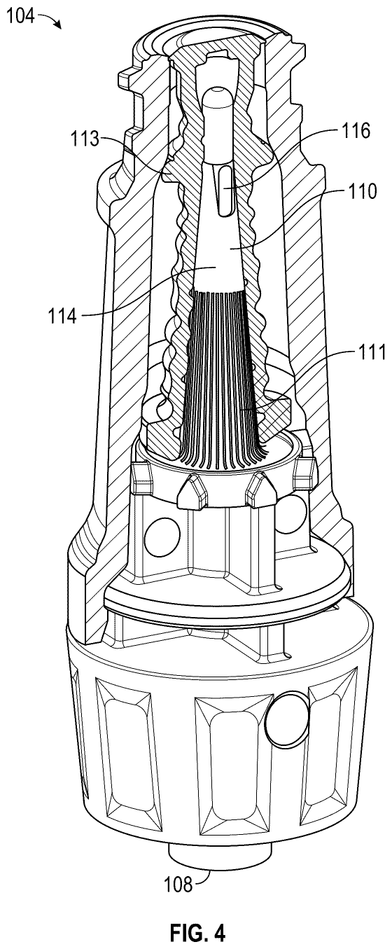

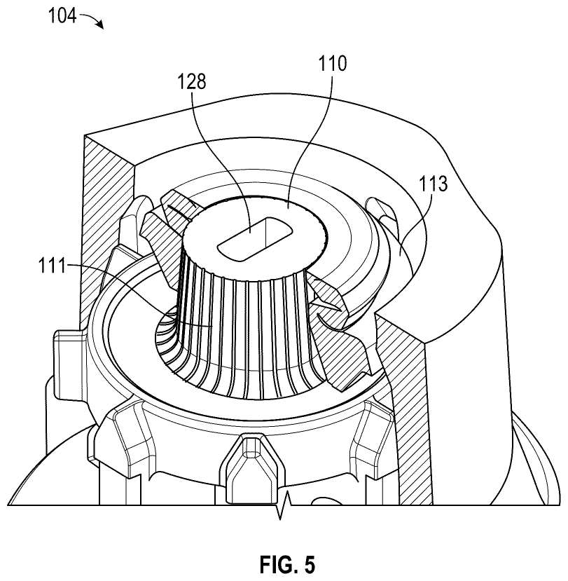

FIGS. 2A and 2B illustrate perspective views of the fluid connector assembly in the uncoupled position and the coupled position, respectively, in accordance with aspects of the present disclosure. In FIG. 2A, the fluid connector assembly 100 is in the uncoupled position. In the uncoupled position, the inlet 112 of the fluid source 102 (e.g., a luer) is not fluidly connected to the outlet 108 of the fluid connector 104. In particular, in the uncoupled position, the sleeve 113 covers the aperture 116 of the post 110 (as shown in FIG. 4), and in this position, fluid cannot pass through the aperture 116 and enter the lumen 128 of the post 110 (shown in FIG. 5).

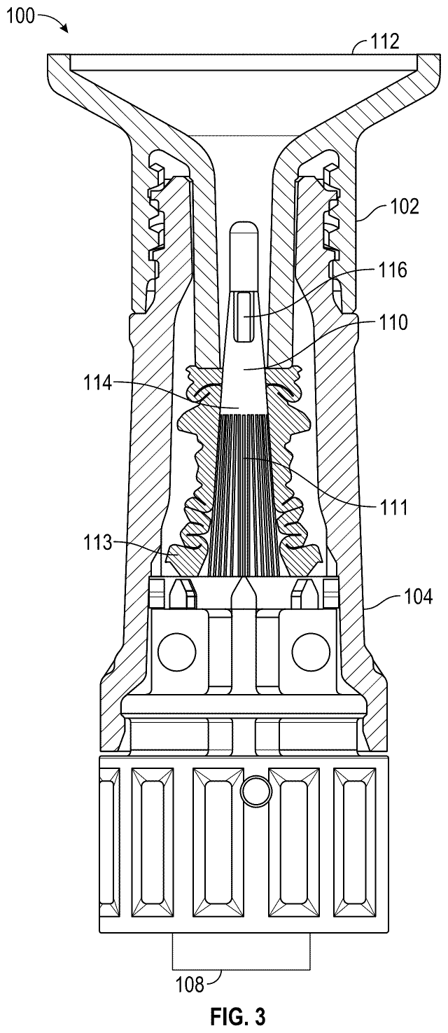

In FIG. 2B, the fluid connector assembly 100 is in the coupled position. In the coupled position, the luer 102 is coupled with the fluid connector 104. In particular, the luer 102 displaces the sleeve 113 toward the outlet 108, which exposes the aperture 116 of the post 110 and fluidly connects the inlet 112 of the luer 102 to the outlet 108 of the fluid connector 104 (as shown in FIG. 3).

FIG. 3 illustrates a cross-sectional view of the fluid connector assembly in the coupled position, in accordance with aspects of the present disclosure. The fluid connector assembly 100 provides a regulated fluid path throughout the components of the fluid connector assembly 100. As shown, the fluid connector assembly 100 includes a luer 102 used as a central body to carry and/or connect with one or more components. In some embodiments, the luer 102 is connected to a medical fluid, such as the medical fluid bag 12 (shown in FIG. 1). The luer 102 may include a hollow, or generally hollow, body that receives one or more components (e.g., the post 110 of the fluid connector 104).

The fluid connector assembly 100 further includes a fluid connector 104 which can couple with the luer 102. The fluid connector 104 may also be referred to as a connector or medical connector. The fluid connector 104 may conform to standards established by the International Organization for Standards (“ISO”) to improve patient safety, minimize medical fluid leakage, and reduce misconnection with other connection devices. In some embodiments, the fluid connector 104 is connected to a catheter line (e.g., tubing 16 in FIG. 1) that delivers the medical fluid to a catheter, such as the catheter 18 (shown in FIG. 1).

The fluid connector 104 includes an outlet 108 that includes a channel fluidly connected to an aperture 116. As a result, the outlet 108 acts as a fluid transmission location for the fluid connector assembly 100. Also, the luer 102 includes an inlet 112 that includes an opening (not shown in FIG. 3). The opening of the inlet 112 acts as a fluid inlet of the fluid connector assembly 100. Accordingly, the opening acts as a fluid receiving location for the fluid connector assembly 100. Each of the outlet 108 and inlet 112 can pass centrally, or at least approximately centrally, through an end of the luer 102 and the fluid connector 104, respectively. Also, each of the outlet 108 and inlet 112 includes a cylindrical, or generally cylindrical, shape. However, other shapes are possible.

The fluid connector 104 includes an aperture 116 formed in a post 110 of fluid connector 104. The post 110 may be a generally conical shape. In some embodiments, the post 110 is a cannula. However, other shapes are possible. The aperture 116 represents a fluid inlet for the fluid connector 104. The aperture 116 is fluidly connected to the outlet 108 via a lumen 128 (shown in FIG. 5).

Additionally, the fluid connector 104 includes a sleeve 113. The sleeve 113 can alternatively be referred to as a valve or compressible valve. The sleeve 113 may cover the aperture 116 to prevent a fluid from entering the lumen 128 of the post 110 (as shown in FIG. 4). The sleeve 113 may extend around the post 110, or in some embodiments may only cover the aperture 116. The sleeve 113 may be made of silicone or another suitable rubber, plastic or other material that is commonly used for IV connecting valves. The sleeve 113 may be secured to the post 110 on a distal end thereof, whereas a proximal end thereof is free to slide relative to the post 110. Accordingly, the sleeve 113 is moveable and can compress or fold by one or more external forces (e.g., a force applied to the sleeve 113 when the luer 102 is coupled to the fluid connector 104) and subsequently return to its original, uncompressed state when an opposite external force is applied.

The sleeve 113 includes a first dimension (shown in FIG. 4) that defines a lengthwise dimension of a major axis of the sleeve 113. When no external force is acting on the sleeve 113 to displace (e.g., compress) the sleeve 113, the lengthwise dimension of the sleeve 113 is defined by the first dimension. Accordingly, the first dimension of the sleeve 113 represents an initial dimension, and the size and shape of the sleeve 113 shown in FIG. 4 represents an initial size and shape. When an external force is acting on the sleeve 113 to displace the sleeve 113, the sleeve 113 reduces to a second dimension (shown in FIG. 3) that is less than the first dimension.

The sleeve 113 is designed to regulate fluid flow through the fluid connector 104. Accordingly, the sleeve 113 acts as a valve for the fluid connector assembly 100. This will be shown in detail below.

As shown in FIG. 3, the post 110 of the fluid connector 104 is at least partially disposed in the luer 102 in the coupled position, causing the proximal end of the sleeve 113 to translate toward the distal end of the sleeve 113. In some embodiments, the sleeve 113 folds at a point between the proximal end and the distal end thereof. In some embodiments, the sleeve 113 rolls upon itself in response to engaging the septum 103.

The displacement of the sleeve 113 causes the aperture 116 of the post 110 to open because the sleeve 113 is no longer covering the aperture 116. As a result, fluid entering the inlet 112 of the luer 102 can further pass through the aperture 116 of the post 110. Accordingly, in the uncovered configuration of the sleeve 113, the outlet 108 is fluidly connected to the inlet 112 through the aperture 116 and the lumen 128. When the sleeve 113 is reduced to the second dimension, a proximal end thereof can engage the luer 102 to prevent decoupling of the fluid connector 104 from the luer 102. In some embodiments, the sleeve 113 can prevent separation of the fluid connector 104 from the luer 102 up to a force of 8 pounds. In some embodiments, the force may be between 3 pounds and 8 pounds. In some embodiments, the force may be between 3 pounds and 5 pounds, and in some embodiments, the force may be about 4 pounds. In some embodiments, the force may be less than or about 3 pounds or more than or about 5 pounds. This separation may prevent the fluid connector assembly 100 from unintended dislodgement or disconnection of the tubing 16 or the catheter 18 from the patient.

When a medical fluid line is connected to the luer 102, the fluid enters the inlet 112 (i.e., fluid inlet) of the luer 102. The fluid can then pass through the inlet 112 and enter the aperture 116 and the lumen 128 (shown in FIG. 5) of the post 110, and subsequently pass through the outlet 108. The fluid can exit the fluid connector assembly 100 through the outlet 108.

Based on the fluid flow through the fluid connector assembly 100, the fluid connector assembly 100 provides neutral fluid displacement in which blood and/or other fluids is/are prevented, or at least substantially limited, from entering a catheter lumen (not shown) positioned in the fluid connector 104 and fluidly connected to the fluid connector assembly 100 during a connector or disconnection between the fluid connector assembly 100 and the catheter lumen, or when the medical fluid line is connected to or disconnected from the luer 102. Based on the features and functionality, the fluid connector assembly 100, unlike positive fluid displacement connector assemblies, does not force fluid into the catheter lumen during a connection or disconnection event. Also, the fluid connector assembly 100, unlike negative fluid displacement connector assemblies, does not allow fluid back into the catheter lumen during a connection or disconnection event. Accordingly, the fluid connector assembly 100 includes a neutral displacement connector assembly.

FIG. 4 illustrates a cross-sectional view of the fluid connector, in accordance with aspects of the present disclosure. The fluid connector 104 has a housing with an inner chamber. The sleeve 113 and the post 110 are located within the inner chamber of the housing.

In FIG. 4, the fluid connector 104 is in the uncoupled position. When the fluid connector 104 is uncoupled from the luer 102, in accordance with aspects of the present disclosure, the sleeve 113 returns to its original shape and size (as shown in FIG. 4) because the sleeve 113 is made of a resilient material. In this regard, the sleeve 113 returns to having the first dimension, representing the original lengthwise dimension of the sleeve 113 prior to displacement by the septum 103. Further, the sleeve 113 covers the aperture 116 of the post 110 to again seal the aperture 116 and prevent fluid passage therethrough. When the fluid connector 104 is in the uncoupled position, the sleeve 113 is in the closed position.

The fluid connector 104 has a post 110 that has a conical or generally conical shape (however, other shapes are also possible). The post 110 has grooves 111 (e.g., ridges, indentations, channels, and/or corrugations). In some embodiments, the grooves 111 are undulating ridges on the surface of the post 110. In some embodiments, the grooves 111 are oriented axially along the surface of the post 110. The grooves 111 can extend from a distal end of the post 110 to a proximal end of the post 110 (i.e., lengthwise or longitudinally) along the surface of the post 110. In some embodiments, the grooves 111 extend circumferentially around the entire length of the post 110. In some embodiments, the grooves 111 extend up the entire length of the post 110. Alternatively, the grooves 111 can extend halfway up the post 110. The grooves 111 can also be confined to the base of the post 110. In some embodiments, the grooves 111 do not start at the base of the post 110, but instead start further up the post 110. In some embodiments, the portion of the post 110 without any grooves comprises a sealing area 114. In some embodiments, the top of the sleeve 113 never reaches the grooves 111 (i.e., the top of the sleeve 113 is never displaced beyond the scaling area 114).

The grooves 111 limit the amount of contact between the post 110 and the valve (i.e., the sleeve 113), which, in turn, restricts the amount of friction between the post 110 and the sleeve 113. The restricted friction helps the sleeve 113 return to the first dimension from the second dimension. In other words, the sleeve 113 can rebound to the closed position and/or return to the proximal end of the post 110 without getting stuck in the open position. The reduced friction also helps the sleeve 113 rebound quickly to the closed position from the open position. When the sleeve 113 returns to the proximal end of the post 110 quickly, the fluid is less likely to leak out of the fluid connector 104.

In some embodiments, the grooves 111 are filled with a lubricant. The lubricant further restricts friction between the sleeve 113 and the post 110. In some embodiments, the sleeve 113 removes and/or displaces the lubricant with respect to the grooves 111 as the sleeve 113 is compressed into the open position or returns to the closed position.

FIG. 5 illustrates a cross-sectional view of the post of the fluid connector, in accordance with aspects of the present disclosure. The fluid connector 104 has a lumen 128 through which fluid can pass when the sleeve 113 is not covering the aperture 116 (i.e., the sleeve 113 is in the open position or the uncovered configuration).

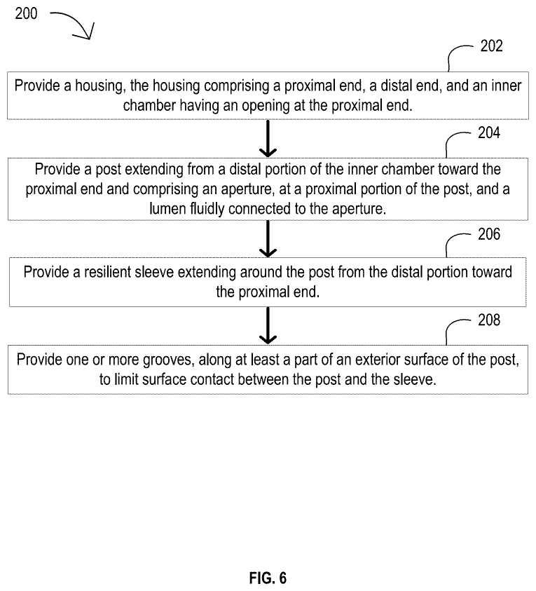

FIG. 6 illustrates a flowchart showing a method for restricting friction in a fluid connector during delivery of a medical fluid, in accordance with aspects of the present disclosure. Fluid connector assemblies shown or described herein can carry out the steps of the method shown in the flowchart 200.

In step 202, a housing is provided. The housing may comprise a proximal end, a distal end, and an inner chamber having an opening at a proximal end of the housing.

In step 204, a post is provided. The post extends from a distal portion of the inner chamber toward the proximal end of the housing. The post comprises an aperture at a proximal portion of the post and a lumen that is fluidly connected to the aperture. In some embodiments, the post has a conical or generally conical shape. Other shapes are also possible.

In step 206, a resilient sleeve is provided. The resilient sleeve extends around the post from the distal portion of the inner chamber toward the proximal end of the housing.

In step 208, one or more grooves are provided along an exterior surface of the post. The grooves can extend along the entire length of the post or along part of the length of the post. The grooves limit surface contact between the post and the sleeve, which restricts friction between the post and the sleeve. A lubricant may be applied to the grooves to further restrict friction between the post and the sleeve.

The fluid connector is configured to form a fluid pathway between the aperture and the lumen when the sleeve is compressed distally to unseal the opening and the aperture. Compressing the sleeve may include displacing a proximal end of the sleeve toward a distal end of the sleeve and permitting fluid to be conducted through the aperture and the lumen between the opening at the proximal end and a distal opening at the distal end.

The features of the present disclosure provide a fluid connector assembly with a sleeve that can be displaced to form a fluid pathway therebetween. When a luer and a fluid connector are coupled together, the luer displaces the sleeve inside the fluid connector and allows fluid flow between the luer and the fluid connector. However, if the luer that displaces the sleeve is separated from the fluid connector, whether unintentionally or intentionally, the fluid pathway the fluid connector assembly closes or is obstructed to prevent fluid loss therefrom, as the sleeve returns to its uncompressed state and closes off the fluid pathway. The features of the present disclosure also provide that upon separation of the luer and the fluid connector, any of the luer and the fluid connector can be cleaned and disinfected, and the luer and the fluid connector can be once again coupled together to cause the sleeve to form a fluid pathway therebetween.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1, clause 8 or clause 15. The other clauses can be presented in a similar manner.

Clause 1. A fluid connector comprising: a housing comprising a proximal end and a distal end, the housing comprising an inner chamber having an opening at the proximal end; a post extending from a distal portion of the inner chamber toward the proximal end and comprising an aperture, at a proximal portion of the post, and a lumen fluidly connected to the aperture; and a resilient sleeve extending around the post from the distal portion toward the proximal end such that the sleeve seals the opening and the aperture in a covered configuration and is compressed distally to unseal the opening and the aperture in an uncovered configuration, wherein the post comprises one or more grooves along at least a part of an exterior surface of the post that limits contact between the post and the sleeve.

Clause 2. The fluid connector system of clause 1, wherein the post is a generally conical shape.

Clause 3. The fluid connector system of clause 1, wherein the post is a cannula.

Clause 4. The fluid connector system of clause 1, wherein the grooves contain a lubricant that restricts friction between the post and the sleeve.

Clause 5. The fluid connector system of clause 1, wherein the grooves comprise undulating ridges on an exterior of the post that restrict friction between the post and the sleeve by limiting surface contact between the post and the sleeve.

Clause 6. The fluid connector system of clause 5, wherein the grooves extend longitudinally from the distal portion of the inner chamber toward the proximal portion of the post.

Clause 7. The fluid connector system of clause 1, wherein the sleeve extends circumferentially around the post.

Clause 8. The fluid connector system of clause 1, wherein the grooves extend circumferentially around the entire post.

Clause 9. The fluid connector system of clause 1, wherein the grooves extend about half an entire length of the post.

Clause 10, wherein in the uncovered configuration, a proximal end of the sleeve is displaced toward a distal end of the sleeve and the fluid connector is configured to permit fluid to be conducted, through the aperture and the lumen, between the opening at the proximal end and a distal opening at the distal end.

Clause 11. A method for restricting friction in a fluid connector during delivery of a medical fluid, the method comprising: providing a housing, the housing comprising a proximal end, a distal end, and an inner chamber having an opening at the proximal end; providing a post extending from a distal portion of the inner chamber toward the proximal end and comprising an aperture, at a proximal portion of the post, and a lumen fluidly connected to the aperture; providing a resilient sleeve extending around the post from the distal portion toward the proximal end; and providing one or more grooves, along at least a part of an exterior surface of the post, to limit surface contact between the post and the sleeve; wherein the fluid connector is configured to form, by compressing the sleeve distally to unseal the opening and the aperture, a fluid pathway between the aperture and the lumen.

Clause 12. The method of clause 11, further comprising applying a lubricant to the grooves to restrict friction between the post and the sleeve.

Clause 13. The method of clause 11, wherein forming a fluid pathway comprises displacing a proximal end of the sleeve toward a distal end of the sleeve such that fluid may be conducted, through the aperture and the lumen, between the opening at the proximal end and a distal opening at the distal end.

### FURTHER CONSIDERATIONS

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear,” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A fluid connector comprising:
a housing comprising a proximal end and a distal end, the housing comprising an inner chamber having an opening at the proximal end;
a post extending from a distal portion of the inner chamber toward the proximal end and comprising an aperture, at a proximal portion of the post, and a lumen fluidly connected to the aperture; and
a resilient sleeve extending around the post from the distal portion toward the proximal end such that the sleeve seals the opening and the aperture in a covered configuration and is compressed distally to unseal the opening and the aperture in an uncovered configuration,
wherein the post comprises one or more grooves along at least a part of an exterior surface of the post that limits contact between the post and the sleeve.

2. The fluid connector of claim 1, wherein the post is a generally conical shape.

3. The fluid connector of claim 1, wherein the post is a cannula.

4. The fluid connector of claim 1, wherein the grooves contain a lubricant that restricts friction between the post and the sleeve.

5. The fluid connector of claim 1, wherein the grooves comprise undulating ridges on an exterior of the post that restrict friction between the post and the sleeve by limiting surface contact between the post and the sleeve.

6. The fluid connector of claim 1, wherein the grooves extend longitudinally from the distal portion of the inner chamber toward the proximal portion of the post.

7. The fluid connector of claim 1, wherein the sleeve extends circumferentially around the post.

8. The fluid connector of claim 1, wherein the grooves extend circumferentially around the entire post.

9. The fluid connector of claim 1, wherein the grooves extend about half an entire length of the post.

10. The fluid connector of claim 1, wherein in the uncovered configuration, a proximal end of the sleeve is displaced toward a distal end of the sleeve and the fluid connector is configured to permit fluid to be conducted, through the aperture and the lumen, between the opening at the proximal end and a distal opening at the distal end.

11. A method for restricting friction in a fluid connector during delivery of a medical fluid, the method comprising:
providing a housing, the housing comprising a proximal end, a distal end, and an inner chamber having an opening at the proximal end;
providing a post extending from a distal portion of the inner chamber toward the proximal end and comprising an aperture, at a proximal portion of the post, and a lumen fluidly connected to the aperture;
providing a resilient sleeve extending around the post from the distal portion toward the proximal end; and
providing one or more grooves, along at least a part of an exterior surface of the post, to limit surface contact between the post and the sleeve;
wherein the fluid connector is configured to form, by compressing the sleeve distally to unseal the opening and the aperture, a fluid pathway between the aperture and the lumen.

12. The method of claim 11, further comprising applying a lubricant to the grooves to restrict friction between the post and the sleeve.

13. The method of claim 11, wherein forming a fluid pathway comprises displacing a proximal end of the sleeve toward a distal end of the sleeve such that fluid may be conducted, through the aperture and the lumen, between the opening at the proximal end and a distal opening at the distal end.

14. A fluid connector comprising:
a housing comprising a proximal end and a distal end, the housing comprising an inner chamber having an opening at the proximal end;
a post, having one or more grooves on an exterior surface, extending in the inner chamber toward the proximal end and comprising an aperture and a lumen fluidly connected to the aperture; and
a resilient sleeve extending around the post such that the sleeve seals the opening and the aperture in a covered configuration and is compressed distally to unseal the opening and the aperture in an uncovered configuration.

15. The fluid connector of claim 14, wherein the post is a generally conical shape.

16. The fluid connector of claim 14, wherein the one or more grooves contains a lubricant that restricts friction between the post and the sleeve.

17. The fluid connector of claim 14, wherein the grooves comprise undulating ridges on an exterior of the post that restrict friction between the post and the sleeve by limiting surface contact between the post and the sleeve.

18. The fluid connector of claim 14, wherein the grooves extend longitudinally from a distal portion of the inner chamber toward a proximal portion of the post.

19. The fluid connector of claim 14, wherein the sleeve extends circumferentially around the post.

20. The fluid connector of claim 14, wherein in the uncovered configuration, a proximal end of the sleeve is displaced toward a distal end of the sleeve and the fluid connector is configured to permit fluid to be conducted, through the aperture and the lumen, between the opening at the proximal end and a distal opening at the distal end.
